(19) United States
(12) Patent Application Publication
(10) Pub. No.: US 2020/0376233 A1
(43) Pub. Date: Dec. 3, 2020

(54) Line Protecting Garment

(71) Applicants: Ricardo Morales, JR., Riverside, CA (US); Estefania Morales, Riverside, CA (US)

(72) Inventors: Ricardo Morales, JR., Riverside, CA (US); Estefania Morales, Riverside, CA (US)

(21) Appl. No.: 16/884,716

(22) Filed: May 27, 2020

Related U.S. Application Data
(60) Provisional application No. 62/853,615, filed on May 28, 2019.

(51) Int. Cl. A61M 25/02; A41F 9/00

(52) U.S. Cl. CPC A61M 25/02; A61M 2205/0216; A61M 2025/0206; A41F 9/00

(57) ABSTRACT

A garment for protecting a line, such as a central line or broviac line, protruding out of a body of a user, the garment having a body strap, and a pocket on an interior side of the body strap to cover and protect the line from the user. The body strap can have a slit through which the line can exit. The garment can have shoulder straps with line traps to further manage the line exiting from the body strap.

### CROSS-REFERENCE TO RELATED APPLICATION

This patent application claims the benefit of U.S. Provisional Patent Application Ser. No. 62/853,615, entitled “Line Protecting Garment,” filed May 28, 2019, which applications is incorporated in its entirety here by this reference.

### TECHNICAL FIELD

This invention relates to a garment or device to prevent or reduce the frequency of user's pulling on lines, such as central lines and broviac lines.

### BACKGROUND

It is an unfortunate situation when young children have to undergo surgical procedures. It is even more unfortunate when such surgical procedures result in medical devices having to be implanted into the child. To make matters even worse, some surgical procedures require medical devices that are exposed and emerge from the patient's body, resulting in the potential for such medical devices to snag or be pulled on which could result in drastic pain and complications for the child.

By way of example only, children with nephrotic syndrome require a central line or a Broviac line (i.e. tubing) to be inserted into their veins or arteries so that healthcare providers can access the bloodstream at any given moment. This is necessary so that the healthcare provider can either inject fluids directly into the bloodstream or collect blood samples for testing.

As difficult as this may be for adults, for a child it is far worse because they do not have the same level of self-control to not pull or yank on the exposed line. In addition, the children are far more active resulting in higher probability of the line getting caught on some object.

Healthcare providers attempt to reduce this probability by excessively taping the line against the child's body. Not only does this cause increased irritation of the skin, but it is also aesthetically unappealing. Furthermore, the taping can come off over time when the child sweats. This results in more taping to keep the line in place.

For the foregoing reasons there is a need for a garment that is aesthetically pleasing, easy to don, easy to wash, and can securely hide a central line or a broviac line in place while still allowing easy exposure for the healthcare provider to access.

### SUMMARY

The present invention is directed to a garment that secures and hides lines, such as central lines and broviac lines that emerge from a user's body, from the reach of a user to protect the lines from being pulled out of the user's body. The garment uses pockets and line traps to keep much of the line hidden from the user. Because the garment is worn like a typical garment, it is aesthetically pleasing, easy to use, and easy to maintain, while reducing the possibility or desire for the user to pull on the lines.

In use, the method for protecting a line emerging from a body of a user comprises inserting a free end of the line into a pocket defined by a line trap and an interior side of a body strap of a garment; extracting the free end of the line from the pocket through an opening of the pocket; routing the free end of the line through a channel defined in a shoulder strap of the garment; and fastening the body strap on the user, whereby the line is protected and inaccessible to the user. In some embodiments, the free end of the line is routed into the channel through an inlet on the interior side of the body strap. Preferably, the free end of the line is routed to a position on top or behind a shoulder of the user to make it more difficult for the user to access.

In some embodiments, the free end of the line is routed into the channel by extracting the free end from the pocket through an opening on the body strap, and inserting the free end of the line into an opening of the channel. Preferably, the free end of the line is accessed by opening a fastener on the channel, and protected by closing the fastener on the channel with the free end of the line tucked into the channel.

### BRIEF DESCRIPTION OF DRAWINGS

FIG. 1 shows a front view of an embodiment of the present invention.

FIG. 2 shows a back view of an embodiment of the present invention.

FIG. 3 shows an exterior side of an embodiment of the present invention laid open.

FIG. 4 shows another embodiment of an exterior side of the present invention laid open with fasteners in the open configuration.

FIG. 5 shows an interior side of an embodiment of the present invention laid open.

FIG. 6 shows another embodiment of an interior side of the present invention laid open with the lining lifted to show features underneath the lining.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention is a line protecting garment 100 designed to protect lines 10, such as broviac and central lines, from being pulled on by those who have lines 10 inserted into their bodies. A line 10 as used in the present invention refers to a medical device that is inserted into a vein or artery of a patient for the purposes of feeding the proper solution, such as fluids, medications, blood products, and the like, as well as drawing blood from the patient. The line comprises a tube 12 or catheter through which fluids pass. The tube 12 has a first end 14 (or insertion end) that is inserted into the patient and a second end 16 (or free end) that emerges from the patient's body and remains external to the patient so as to be accessible by the healthcare provider. The line can further comprise a needle at the first end 14 of the tube to facilitate insertion, and a cap 18 at the second end of the tube to facilitate connection to other medical devices that are used in conjunction with the line 10.

Typically, the first end 14 of the tube 12 is inserted into the patient's body. The tube 12 then emerges from an incision in the patient's body, such as through the chest. As such, the patient has a tube 12 dangling from his or her chest. Left alone, the patient can pull on the line 10 essentially creating foreseeable complications. The line protecting garment 100 of the present invention is designed to reduce the probability of the patient being able to pull on the line 10.

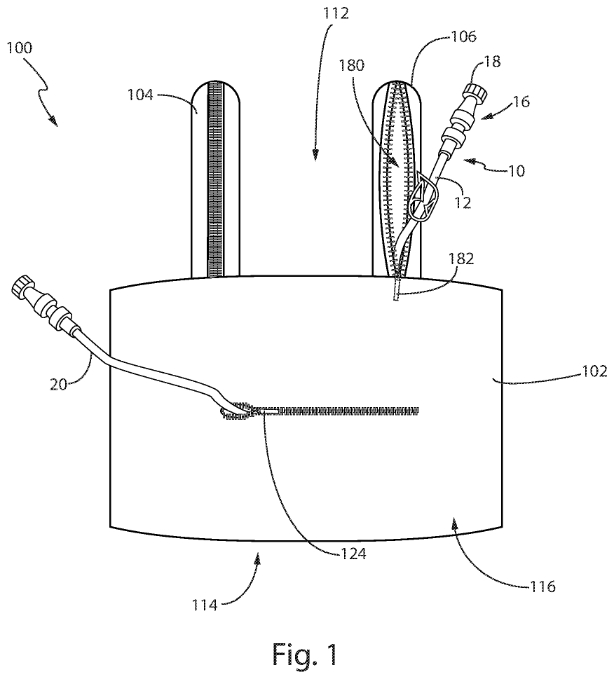

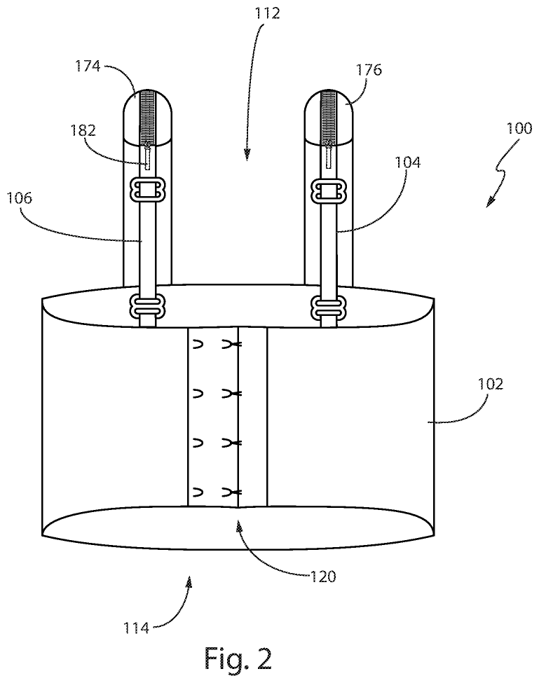

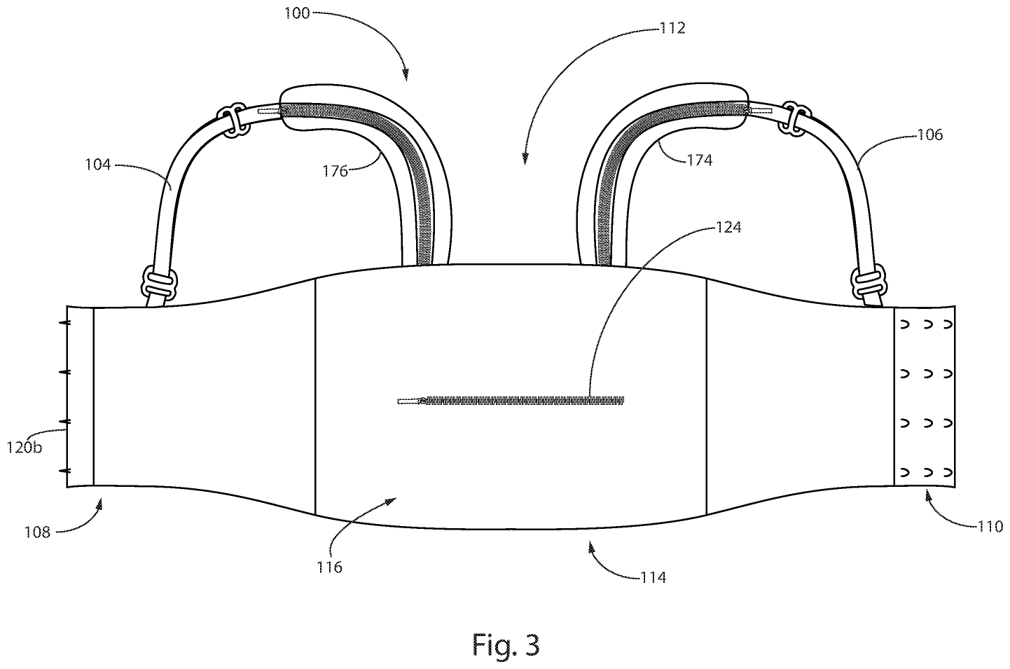

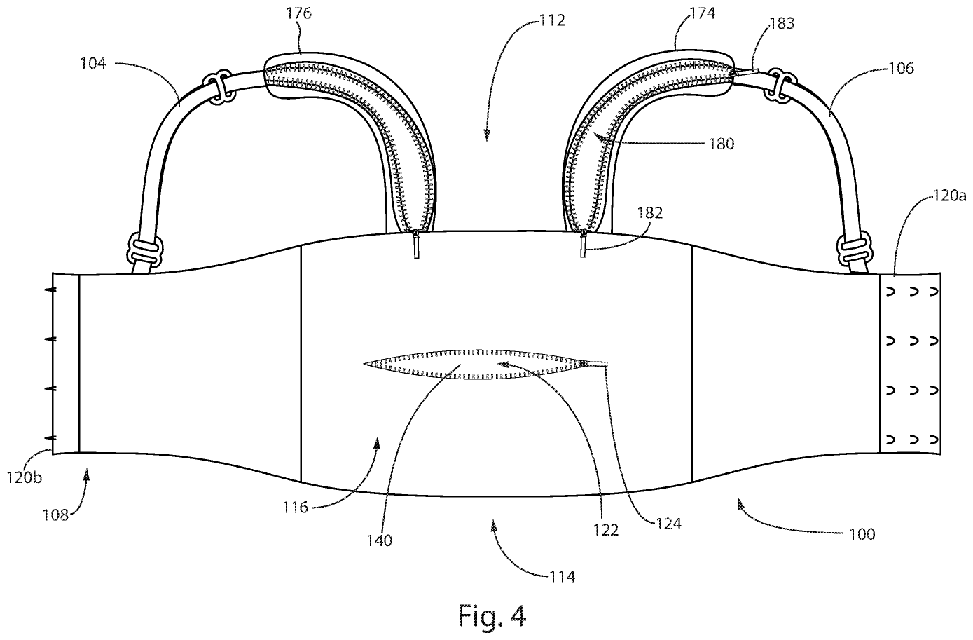

With reference to FIGS. 1 and 2, the line protecting garment 100 comprises a body strap 102. In some embodiments, the line protecting garment 100 further comprises shoulder straps 104, 106. The body strap 102 is configured to wrap around a user's body, such as around the chest area, the abdomen area, or both. As such, in the preferred embodiment, the body strap 102, when laid open as shown in FIGS. 3-6 may be an elongated strip of material generally rectangular in shape having a first lateral side 108, a second lateral side 110 opposite the first lateral side 108, a top side 112 adjacent to the first lateral side 108 and the second lateral side 110, and a bottom side 114 opposite the top side 112, and adjacent to the first lateral side 108 and the second lateral side 110. The first lateral side 108, second lateral side 110, top side 112, and bottom side 114 define the bounds of an exterior side 116 and an interior side 118 opposite the exterior side 116. The interior side 118 is applied to the user's body, and the exterior side 116 is the side visible when the line protecting garment 100 is properly worn by the user.

In some embodiments, the first lateral side 108 and second lateral side 110 can be fastened together with a fastener 120. For example, the first lateral side 108 can comprise a first partial fastener 120a, and the second lateral side 110 can comprise a second partial fastener 120b to bind with the first partial fastener 120a to complete the fastener 120. For example, the first partial fastener 120a and the second partial fastener 120b can be hooks and loops, zippers, buttons, hook and loop fasteners, and the like, or any combination thereof. In some embodiments, the fastener 120 may be a tie, such as a cord, string, rope, cable, chain, and the like, that can tie the first lateral side 108 to the second lateral side 110. In such embodiments, the first lateral side 108 and the second lateral side 110 may be lined with eyelets, with or without grommets for reinforcement, through which the tie can be weaved.

In some embodiments, the first lateral side 108 and the second lateral side 110 can be integrally formed together as a single unit. Therefore, a fastener 120 may not necessarily be required, although one still can be used as appropriate.

Preferably, the line protecting garment 100 comprises elastic material. When properly worn, the elastic material creates a radially inwardly directed compressive force against the user's body. This compressive force keeps the line close to the user's body. It also provides comfort for the user as the line protecting garment 100 exhibits flexibility to move with the user. The line protecting garment 100 can comprise materials such as cotton, polyester, spandex, nylon, and any other textile material, or any combination thereof. For example, the line protecting garment can be 50 percent cotton and 50 percent polyester. The line protecting garment 100 does not necessarily have to be elastic. Other materials that are not known for elasticity can be used.

Preferably, the exterior side 116 of the garment 100 comprises an opening 122. Preferably, the opening 122 is in the form of a narrow slit. The opening 122 can be made closeable using any fastener 124 as described above. Preferably, the fastener 144 is a zipper. When the slit is unfastened an access is created to the interior side 118 from the exterior side 116.

The interior side 118 comprises a line trap 126. The line trap 126 contains the line 10 and properly routes the line 10 from the interior side 118 to the outside of the garment 100 where the line 10 can be accessible by a healthcare provider. The line trap 126 can be a patch of material fastened to the interior side 118 of the garment 100. In the preferred embodiment, the patch has a first lateral side 130, a second lateral side 132 opposite the first lateral side 130, a top side 134 adjacent to the first lateral side 130 and the second lateral side 132, and a bottom side opposite 136 the top side 134 and adjacent to the first lateral side 130 and second lateral side 132. The first lateral side 130, second lateral side 132, the top side 134, and the bottom side 136 define the bounds of a first surface 138 of the patch and a second surface 140 of the patch opposite the first surface 138. The first lateral side 130 terminates at a first lateral edge 142, the second lateral side 132 terminates at a second lateral edge 144, the top side 134 terminates at a top edge 146, and the bottom side 136 terminates at a bottom edge 148.

The second surface 140 of the line trap 126 can be placed against the interior side 118 of the garment 100. Preferably, the line trap 126 is placed against the interior side 118 to overlap with the opening 122. In other words, when viewing the garment 100 from the interior side 118 with the patch properly applied, the opening 122 can be covered by the patch. The size of the patch is smaller than the size of the interior surface 118. Therefore, the entire patch fits within the interior surface 118.

At least two sides of the patch can be fastened to the interior side 118 of the garment 100 so as to create a space in between the line trap 126 and the interior surface 118. Preferably, the two sides of the patch fastened to the interior surface 118 are fastened along their respective edges. For example, the first lateral edge 142 and the second lateral edge 144 may be fastened to the interior surface 118. In some embodiments the top edge 146 and the bottom edge 148 may be fastened to the interior surface 118. In some embodiments, three of the edges may be fastened to the interior surface 118. For example, the top edge 146, the bottom edge 148, and one of the first or second lateral edges 142, 144 can be fastened to the interior surface 118. Alternatively, the first lateral edge 142, the second lateral edge 144, and one of the top or bottom edges 146, 148 can be fastened to the interior surface 118. In any of these cases, at least one opening 150 is created along one of the edges.

Having an opening 150 in between the line trap 126 and interior surface 118 provides access to the space in between the line trap 126 and the interior surface 118, creating a pocket. The free end 16 of the line 10 can then be inserted into the pocket through the opening 150. From the pocket, the free end 16 of the line 10 can be passed through the opening 122 to exit the pocket onto the exterior side 116 of the body strap 102 where the free end 16 of the line 10 can be routed to a position that would be least accessible or difficult to access by the user. For example, the free end 16 of the line 10 can be routed over the shoulder of the user towards his or her back side.

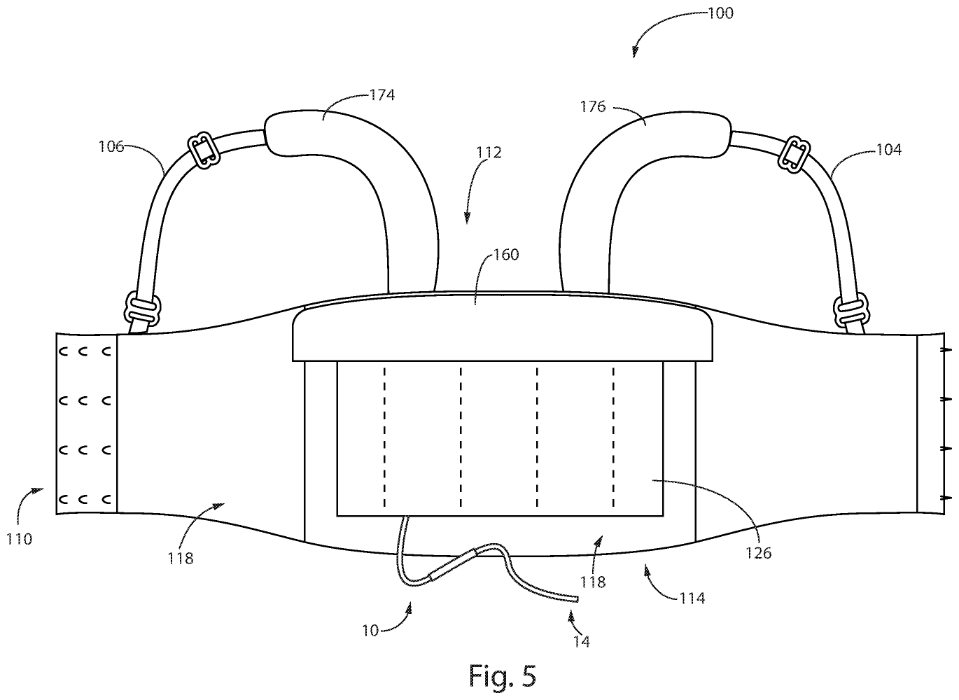

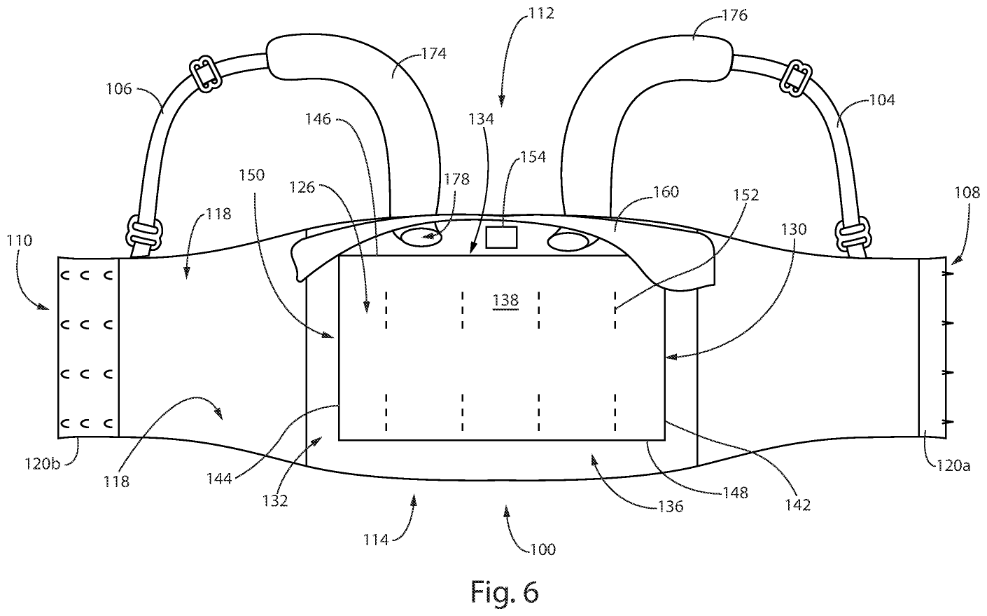

In some instances, users may have multiple lines 10, 20 to manage. For example, a central line exiting from the user's body may have a branch with multiple lines 10, 20 connected to the branch. In some instances, users may have multiple lines 10, 20 exiting from their bodies. As such, the line protecting garment 100 can have multiple discrete pockets on the interior side 118. For example, the patch can be fastened to the interior side 118 of the body strap 102 in a manner that creates multiple discrete pockets. In some embodiments, a portion of the patch can be fastened to the interior side 118 of the body strap 102 in a direction from the bottom side 136 of the patch towards the top side 134 of the patch, referred to as a vertically fastened line 152. Preferably, the patch can have multiple vertically fastened lines intermittently spaced apart to create the multiple pockets in the vertical orientation (meaning the opening(s) to the pocket(s) faces the top side and/or bottom side) as shown in FIGS. 5 and 6. Therefore, in embodiments with vertically fastened lines 152, the first lateral side 130 and the second lateral side 132 of the patch can be fastened to the interior side 118 of the body strap 102, and the top side 134, the bottom side 136, or both the top and bottom sides 134, 136 of the patch can remain open.

In some embodiments, a portion of the patch can be fastened to the interior side 118 of the body strap in a direction from the first lateral side 130 of the patch towards the second lateral side 132 of the patch, referred to as a horizontally fastened line. Preferably, the patch can have multiple horizontally fastened lines intermittently spaced apart from the bottom side 136 to the top side 134 to create the multiple pockets arranged in the horizontal orientation (meaning the opening(s) to the pocket(s) faces the first and/or the second lateral sides). Therefore, in embodiments with horizontally fastened lines, the top side 134 and the bottom side 136 of the patch can be fastened to the interior side 118 of the body strap 102, and the first lateral side 130, the second lateral side 132, or both the first and second lateral sides 130, 132 of the patch can remain open. In other words, the vertically fastened lines 152 shown in FIGS. 5 and 6 would be rotated 90 degrees. In some embodiments, the fastened lines creating the pockets between the line trap 126 and the body strap 102 could be oblique lines having some angle in between the vertical fastened lines 152 and the horizontal fastened lines. In some embodiments, the fastened lines do not have to be straight lines, but rather can be curved, or change directions.

In some embodiments, to further manage the lines 10, 20 trapped in any of the pockets, the body garment can further comprise a loop 154 on the interior side 118. The loop 154 for can be positioned adjacent to any of the openings of the pocket. For example, the loop 154 can be fastened inside the pocket, outside the pocket, on the patch or on the interior side 118 of the body strap 102. As the line 10 is inserted into the pocket, or immediately after entering the pocket, the line can be routed through the loop 154. Similarly, as the line 10 exits the pocket, or immediately before exiting the pocket, the line 10 can be routed through the loop 154. Routing the line 10 through the loop 154 further prevents the line 10 from moving around inside the pocket. Preferably, the loop 154 is an elastic loop so as to create a compressive force against the line 10 to further enhance the securing feature of the loop 154.

At times, the interior side 118 of the body strap 102 can be irritating or uncomfortable to the user, particularly where there are fasteners that can rub against the user's skin. In addition, in some embodiments, portions of the line 10 or components of the line 10 may rub against the user's skin even with the pocket present. As such, in some embodiments, as shown in FIGS. 5-6, the body strap 102 can comprise a lining 160 on the interior side 118. The lining 160 can be fastened to the interior side 118 in a manner that covers portions of the garment that cause irritation or discomfort to the user. As such, the lining 160 can cover a portion of the interior side 118 or substantially all of the interior side 118, including a portion of the pocket, or substantially all of the pocket. The lining 160 can be made of any material that is comfortable on the skin.

In some embodiments, the lining 160 can be a padding. As such, the lining 160 can be material that is thicker than the material used for the body strap 102, or the lining 160 can contain material that is thicker than the material used for the body strap 102. By using a lining 160 that functions as a padding, the user is less likely to feel the lines against his or her body. The lining 160 can be fastened to the interior side 118 of the body strap 102 in a partially removable or completely removable manner. For example, the lining 160 can be fixed along one of the sides, and attached with removable fasteners (e.g. hook and loop fasteners, zippers, buttons, and the like) along the remaining three sides. This creates a flap that can be closed on top of the pockets.

In some embodiments, the lining 160 can be positioned along the side where most irritation may occur. For example, if the line 10 is routed out adjacent the top side 112, the lining 160 can be fastened adjacent the top side 112. The lining 160 can be fastened along the top edge and adjacent to the sides of the pocket to create a hood along the top side 112 of the body strap 102 to enclose any exposed lines that might be protruding from the top side 112.

In some embodiments, the line protecting garment 100 further comprises shoulder straps 104, 106, preferably, adjustable shoulder straps. The shoulder straps 104, 106 help to keep the body strap 102 secured to the user's body by supporting the body strap 102 using the shoulders of the user, the way bra straps and suspenders function. The shoulder straps 104, 106 of the present invention, however, further comprise channels 174, 176. Each shoulder strap 104, 106 can have its own channel 174, 176. The channels 174, 176 allow the lines to pass through the shoulder straps 170, 172 in a hidden manner. After the free end 16 of the line 10 exits the pocket or the opening 122, the free end 16 can be fed into one of the channels 174, 176. The channels 174, 176 can route the line 10 onto the user's shoulder making the line 10 less accessible to the user.

The following description describes a single channel 174; however, the description herein applies to both channels 174, 176. The channel 174 has an inlet 178 through which the line can be inserted into the channel 174, and an opening 180 through which the line 10 can exit the channel 174. In some embodiments, the inlet 178 of the channel 174 is positioned on the interior side 118 of the body strap 102. This configuration maximizes the amount of line 10 that is hidden from the user. Thus, when the line protecting garment 100 is donned by the user, it is positioned over the portion of the user's body where the line 10 exits the body. This covers the line 10 when it exits the body. The line 10 is routed through the line trap 126 keeping the line 10 covered. After exiting the line trap 126, the line 10 is routed through the channel 174, which continues to keep the line 10 covered. Only where the line 10 exits the opening 180 of the channel 174 is the line exposed.

The opening 180 of the channel 104 can be positioned at a point furthest from the body strap 102. For example, in some embodiments, the channel 174 may extend from the body strap 102 to the apex of the shoulder of the user. As such, the line 10 can exit at the apex of the shoulder of the user. In some embodiments, the channel 174 may extend further rearward towards the back of the user. As such, the line 10 can exit behind (rearward) the user making the free end of the line inaccessible to the user.

In some embodiments, the opening 180 of the channel 174 can be closed. As such, the opening 180 of the channel 174 may be lined with a fastener 182, such as those discussed herein. In one embodiment, the opening 180 of the channel 174 may extend substantially the length of the channel 174. A zipper can be used to close the channel 174. Preferably, a two-way zipper 182, 183 is used so that the line can be secured at any position along the channel 174. In some embodiments, the opening 180 of the channel 174 may be a hole substantially the size of the line to allow the line to exit the channel 174.

In some instances, users with lines 10 implanted in their bodies undergo additional medical procedures that can include imaging. Metal objects can interfere with some imaging devices. As such, in the preferred embodiment, the line protecting garment 100 is made without any metal components. For example, although the fasteners can be made of any material, preferably, the fasteners are made of non-metallic material, such as plastic, nylon, and other fabrics.

The body strap 102 can be made of any material that is comfortable on the skin, while being capable of securing the line against the user's body. The body strap 102 can be resistant to staining or coated to prevent staining. The body strap 102 is preferably made of breathable material. Preferably, the body strap 102 comprises elastic material. Using elastic material allows the line 10 to be compressed against the user's body while still offering some freedom in movement. Inelastic material can also be used; however, there may be reduced freedom in movement when the body strap 102 is secured to the user tight enough to keep the line trapped. The body strap 102 can have fasteners that are adjustable so as to adjust the sizing of the body strap 102 for improved comfort or mobility.

In use, the user undergoes a surgical procedure in which the insertion end 14 of the line 10 is surgically implanted into his or her body. At the surgical incision site where the line 10 exits the user's body, a portion of the line is taped or strapped against the user's body according to standard procedure. Then, the line protecting garment 100 is put on the user in a manner that the body strap 102 covers the surgical incision site. Because of the compressive force against the line 10, less tape is required to secure the line 10 at the surgical incision site. Using less tape at the surgical incision site creates less irritation on the skin as the tape can be irritating to the skin.

The remaining line 10 is routed into the line trap 126. In situations where there are multiple lines, each line 10, 20 may be routed through its own respective pocket of the line trap 126. In some embodiments, the free end 16 of the line 10 can then be pulled out of the pocket and through the opening 122 to pass from the interior side 118 of the garment 100 to the exterior side 116. The line 10 can then be routed over the shoulder or to a convenient location as determined by the healthcare provider. Alternatively, the line can be directed into one of the channels 174, 176 through the inlet 178 and out the opening 180, for example, over or behind (rearward) the shoulder.

In some embodiments, rather than the free end 16 of the line 10 exiting through the opening 122 of the body strap 102, the free end 16 of the line 10 can enter through the inlet 178 of the channel 174, 176 from the interior side 118 of the body strap 102. The line can then route through the channel 174, 176 up onto the shoulder of the user and exit through the opening 180. As such, most of the line 10 is hidden from the user.

The lateral sides 108, 110 of the body strap 102 are pulled behind the user and fastened to each other using a fastener 120, such as those described herein. The fasteners 120 can be adjustable so as to create a tight fit against the user's body so that the line 10 is compressed against the user's body.

In some embodiments, during everyday activity, the free end 16 of the line 10 can remain hidden inside the channel 174, 176. When the user sees his or her healthcare provider for treatment, the healthcare provider opens the channel 174, 76, exposes the free end 16 of the line 10, and performs the necessary medical procedure with the line. When the healthcare provider is finished, the free end of the line is placed back into the channel 174, 176 where it can be safely stored.

In some embodiments, the free end 16 of the line 10 can be stored inside the line trap 126. When the user sees his or her health care provider for treatment, the healthcare provider opens the opening 122 on the exterior side 116 of the body strap 102, exposes the free end 16 of the line, and performs the necessary medical procedure with the line 10. When the healthcare provider is finished, the free end 16 of the line 10 is placed back into the line 126 where it can be safely stored.

When the time comes to wash the line protecting garment 100, the body strap 102 can be easily removed by unfastening the fasteners 120 on the back, removing the line 10 from the line protecting garment 100, and placing the line protecting garment 100 into the wash. Because the line protecting garment 100 looks like typical clothing, it is also aesthetically pleasing to the eye.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

What is claimed is:

1. A garment, comprising:
a) a body strap, comprising
(i) a first lateral side,
(ii) a second lateral side opposite the first lateral side,
(iii) a top side adjacent to the first lateral side and the second lateral side, and
(iv) a bottom side opposite the top side, and adjacent to the first lateral side and the second lateral side,
(v) an exterior side defined by the first lateral side, second lateral side, top side, and bottom side,
(vi) an interior side opposite the exterior side and defined by the first lateral side, second lateral side, top side, and bottom side,
(vii) a first fastener for connecting the first lateral side and the second lateral side,
(viii) a line trap connected to the interior side and defining at least one pocket in between the line trap and the interior side,
(ix) a lining on the interior side overlapping on the at least one pocket,
(x) wherein the body strap comprises elastic material,
(xi) wherein the body strap comprises a first opening to access the interior side from the exterior side, the first opening having a second fastener to close the first opening,
(xii) wherein the line trap and the interior side define a second opening to access the at least one pocket between the line trap and the interior side; and
b) a pair of shoulder straps, wherein at least a first shoulder strap of the pair of shoulder straps defines a channel having an opening, the channel defining a length, the first shoulder strap comprising a third fastener to close the opening of the channel, wherein the channel further defines an inlet positioned on the interior side of the body strap.

2. A garment, comprising:
a) a body strap, comprising an exterior side and an interior side;
b) a line trap connected to the interior side of the body strap and defining a space in between the line trap and the interior side of the body strap, the line trap defining a first opening and a second opening, the first and second openings configured to access the space in between the line trap and the interior side; and
c) a pair of shoulder straps.

3. The garment of claim 2, wherein the body strap comprises a third opening through the exterior side to the interior side.

4. The garment of claim 3, wherein the third opening is positioned on the body strap to access the space in between the line trap and the interior side.

5. The garment of claim 4, wherein at least a first shoulder strap of the pair of shoulder straps defines a channel having an inlet and an opening, the inlet adjacent to the body strap.

6. The garment of claim 5, wherein the opening of the channel is positioned at an apex of the first shoulder strap or further rearward.

7. The garment of claim 5, wherein the channel further comprises a fastener to open and close the channel.

8. The garment of claim 5, wherein the first shoulder strap, wherein the inlet of the channel is on the interior side adjacent to the first or second opening of the line trap.

9. The garment of claim 2, wherein at least a first shoulder strap of the pair of shoulder straps defines a channel having an inlet and an opening, the inlet adjacent to the body strap.

10. The garment of claim 9, wherein the inlet is positioned on the interior side adjacent to the first or second opening of the line trap.

11. The garment of claim 10, wherein the opening of the channel is positioned at an apex of the first shoulder strap or further rearward.

12. The garment of claim 9, wherein the channel further comprises a fastener to open and close the channel.

13. The garment of claim 2, further comprising a loop positioned in between the second opening of the line trap and the inlet of the channel.

14. The garment of claim 2, further comprising a lining on the interior side of the body strap.

15. The garment of claim 14, wherein the lining is a padding.

16. A method for protecting a line emerging from a body of a user, comprising:
a) inserting a free end of the line into a pocket defined by a line trap and an interior side of a body strap of a garment;
b) extracting the free end of the line from the pocket through an opening of the pocket;
c) routing the free end of the line through a channel defined in a shoulder strap of the garment; and
d) fastening the body strap on the user, whereby the line is protected and inaccessible to the user.

17. The method of claim 16, wherein the free end of the line is routed into the channel through an inlet on the interior side of the body strap.

18. The method of claim 17, wherein the free end of the line is routed to a position on top or behind a shoulder of the user.

19. The method of claim 16, wherein the free end of the line is routed into the channel by extracting the free end from the pocket through an opening on the body strap, and inserting the free end of the line into an opening of the channel.

20. The method of claim 16, further comprising accessing the free end of the line by opening a fastener on the channel.
